(19) United States
(12) Patent Application Publication
(10) Pub. No.: US 2015/0174097 A1
(43) Pub. Date: Jun. 25, 2015

(54) TREATING EWING'S SARCOMA AND EWS-FLI1 RELATED DISORDERS

(71) Applicant: STAR BIOTECHNOLOGY, LLC, Tiburon, CA (US)

(72) Inventors: Ramani Aiyer, San Jose, CA (US); James E. O'Dorisio, Tiburon, CA (US)

(73) Assignee: Star Biotechnology, LLC, Tiburon, CA (US)

(21) Appl. No.: 14/415,705

(22) PCT Filed: Jul. 18, 2013
(86) PCT No.: PCT/US2013/051105

§ 371 Date: Jan. 19, 2015

Related U.S. Application Data
(60) Provisional application No. 61/674,047, filed on Jul. 20, 2012.

(51) Int. Cl. A61K 31/404; A61K 47/22; A61K 9/00; A61K 47/10

(52) U.S. Cl. CPC A61K 31/404; A61K 47/10; A61K 47/22; A61K 9/0053; A61K 9/0019

(57) ABSTRACT

The field relates to compositions and methods for treating Ewing's Sarcoma and other disorders related to EWS-FLI1.

### CROSS-REFERENCE TO RELATED APPLICATIONS

This application claims the benefit of U.S. Provisional Patent Application No. 61/647,047 filed Jul. 20, 2012, the entirety of which application is herein incorporated by reference.

### FIELD

The field relates to compositions and methods for treating Ewing's Sarcoma and other disorders related to EWS-FLI1.

### BACKGROUND

The compound 4,7-Dichloro-1,3-dihydro-3-hydroxy-3[2-(4-methoxyphenyl)-2-oxoethyl]-2H-indol-2-one (the compound of Formula I) has been reported as an antagonist of RNA helicase binding to the oncogenic protein EWS-FLI1. However, this compound is highly insoluble in water. Accordingly, there is a need for a formulation of this compound which has increased aqueous solubility for pharmaceutical applications and furthermore there is a need for effective dosing and administration regimens for this compound.

### SUMMARY

In an embodiment, is provided herein, a pharmaceutical composition, comprising the compound of Formula I:

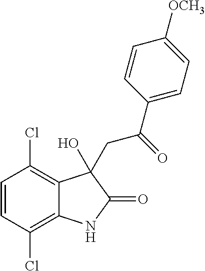 [SMILES: COc1ccc(C(=O)CC2(O)C(=O)Nc3c(Cl)ccc(Cl)c32)cc1]

or a pharmaceutically acceptable salt thereof in an amount effective to treat Ewing's Sarcoma in a subject in need thereof, and further comprising an organic solvent, an organic co-solvent and a lipid in a pharmaceutically acceptable aqueous liquid unit dosage form.

In a further embodiment, is provided herein a method for treating Ewing's sarcoma in a subject in need thereof, comprising administering to the subject at least once a day for at least 1.5 days, the compound of Formula I or a pharmaceutically acceptable salt thereof.

In a further embodiment, is provided herein a method Ewing's sarcoma in a subject in need thereof, comprising administering to the subject the compound of Formula I or a pharmaceutically acceptable salt thereof such that plasma levels of at least 1 micromolar (μM) or 366 nanograms (ng)/mL of the compound of Formula I are sustained in the subject's plasma for at least 36 hours.

### BRIEF DESCRIPTION OF THE DRAWINGS

FIGS. 1A and 1B depict data of pharmacokinetic studies in CF1 mice with respect to administration of the API in the formulation shown in the Figures and via the route of administration shown in the Figures.

FIGS. 2A and 2B depict data of pharmacokinetic studies in CF1 mice with respect to administration of the API in the formulation shown in the Figures and via the route of administration shown in the Figures.

FIG. 3 depicts data obtained from a TC71Luc Ewing's sarcoma xenograft murine model for mice treated with the API in an EtOH (5%), PEG400 (40%), PBS formulation. The x-axis is time post-administration of the API in days with 0 being the first day of administration of API. The y-axis is tumor volume in mm³.

FIGS. 4A and 4B depict IC₅₀ data obtained from in vitro cytotoxicity assays using TC71Luc Ewing's sarcoma cell lines in the presence of the compound of Formula I in an EtOH (5%), PEG400 (40%), PBS formulation. The graphs show the effect of contact with different doses of the compound of Formula I on TC71 cell line cells. The y-axis represents the amount of viable cells. The x-axis shows the different doses of the compound of Formula I. “Parental” cells are compared to “selected” cells. The “selected” cells differ from the “parental” cells in that the “selected” cells have been made resistant to the cytotoxic effect of the compound of Formula I. The tables in FIGS. 4A and 4B show IC₅₀ values for the cyto-toxicity assays.

The following abbreviations are used in the Figures:

IV=intravenous administration

PO=oral administration

JARAM-2012 (also abbreviated herein as JARAM)=the compound of Formula I

IP=intraperitoneal administration

_X=administration for X number of times per week (i.e., 1X means 1 time per week, 3X means 3 times per week, 5X means 5 times per week)

/w or /W=per week.

### DETAILED DESCRIPTION OF THE INVENTION

Herein are described compositions and methods for treating Ewing's Sarcoma. Further described herein are compositions and methods for treating disorders related to the oncogenic protein EWS-FLI1.

The term “unit dosage form” refers to a physically discrete unit suitable for dosing a subject, i.e., each unit containing a predetermined quantity of the active pharmaceutical ingredient calculated to produce the desired therapeutic effect either alone or in combination with one or more additional units.

The term “subject” refers to an animal in need of the treatment or prophylactic methods described herein, for example, a human patient or an animal or mammal in need of veterinarian treatment or prevention.

In an embodiment, the compositions and methods herein are directed to a compound of Formula I:

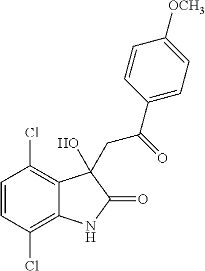 [SMILES: COc1ccc(C(=O)CC2(O)C(=O)Nc3c(Cl)ccc(Cl)c32)cc1]

4,7-Dichloro-1,3-dihydro-3-hydroxy-3[2-(4-methoxyphenyl)-2-oxoethyl]-2H-indol-2-one

In a further embodiment, the compositions and methods herein are directed to sterioisomers, metabolites, prodrugs, derivatives, solvates and pharmaceutically acceptable salts of the compound of Formula I (each of the foregoing, collectively, referred to herein as the “active pharmaceutical ingredient” or the “API”) and pharmaceutical compositions comprising the API and other ingredients in various dosage forms. As is of course understood, all the compositions and methods herein may be administered in aqueous liquid unit dosage forms.

Pharmaceutically acceptable salts are organic and inorganic salts chosen, for example, based on physical and chemical stability, flowability, hydro-scopicity, solubility and other factors. Examples of pharmaceutically acceptable salts are hydrochloride, hydrobromide, sulfphate, tartate, mesylate, maleate and citrate. By way of further illustration, pharmaceutically acceptable salts are described in the Handbook of Pharmaceutical Salts Properties, Selection, and Use (1st edition, Wiley) (2002).

Further embodiments are directed to pharmaceutical compositions comprising the API together with pharmaceutically acceptable excipients. Examples of pharmaceutically acceptable excipients include: anti-adherents, lubricants, glidants, binders, coatings, enterics, disintegrants, emulsifiers, fillers, lubricants, buffers (or other pH adjusting agents), sorbents, buffers, preservatives, sweetners, flavors and colors. Antiadherents, lubricants and glidents are used, for example, to reduce adhesion during manufacturing of pharmaceutical compositions. An example of an antiadherent is magnesium stearate. Oils are an example of lubricants. Binders are used, for example, to hold together dose ingredients, to enhance mechanical strength and to provide volume to low active pharmaceutical ingredient dosage forms. Examples of binders are saccharides, gelatins and polymers. Enterics are used, for example, to modify drug release characteristics. Emulsifiers and solubilizers are used, for example, to improve solubility or improve stability of a drug containing emulsion. Examples of emulsifiers are surfactants, for example, polysorbates. Fillers are used, for example, as bulking agents and numerous vegetable fats and oils can be used as fillers, for example, in soft gelatin capsules. Disentegrants are used, for example, to provide for release of active pharmaceutical ingredient, when the pharmaceutical composition is in a desired environment, for example in the aqueous environment of the digestive tract. Examples of disintegrants includes cross-linked polymers and modified starches.

Pharmaceutical compositions may also be formulated to provide slow, controlled or sustained release of the active agent using, by way of example, hydroxypropyl methyl cellulose in varying proportions or other polymer matrices, liposomes and/or microspheres. In addition, the pharmaceutical compositions described herein may contain opacifying agents and may be formulated so that they release the active agent only, or preferentially, in a certain portion of the gastrointestinal tract, optionally, in a delayed manner. The active agent can also be in micro-encapsulated form, if appropriate, with one or more of the above-described excipients.

The pharmaceutical compositions described herein may be administered, for example, by the following administration routes: oral, bucal, mucosal, nasal, transdermal, pulmonary, parenteral, rectal, subcutaneous, intravenous, and intramuscular.

Furthermore, the pharmaceutical compositions described herein can be in, for example, solid, liquid, or semi-solid dosage forms. For oral administration, if in a liquid, or semi-solid dosage form, the dosage form can be: a syrup, emulsion, soft gelatin capsule or injectable liquid such as an aqueous or non-aqueous liquid suspension or solution. Further suitable liquid dosage forms for oral administration include, for example, pharmaceutically acceptable emulsions, microemulsions, solutions, suspensions, syrups and elixirs. Liquid dosage forms can comprise the active agent and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof. Suspensions can contain suspending agents such as, for example, ethoxylated isostearyl alcohols, polyoxyethylene sorbitol and sorbitan esters, microcrystalline cellulose, aluminium metahydroxide, bentonite, agar-agar and tragacanth, and mixtures thereof.

For parenteral administration (for example, by subcutaneous, intravenous, intramuscular, or intraperitoneal injection), the pharmaceutical compositions described herein can be administered by injection, for example, in solution, suspension, or emulsion. Exemplary solvents for preparing such formulations include water, saline, low molecular weight alcohols such as propylene glycol, polyethylene glycol, oils, gelatin, fatty acid esters such as ethyl oleate, and the like. Parenteral formulations can also contain one or more anti-oxidants, solubilizers, stabilizers, preservatives, wetting agents, emulsifiers, and dispersing agents. Surfactants, additional stabilizing agents or pH-adjusting agents (acids, bases or buffers) and anti-oxidants can also be included in the parenteral injectable. Moreover, the injectable administration may be in a form so that absorption from the injection site is gradual or over a prolonged period, for example, a depot injection. In further embodiments, the injectable dosage forms are sterile and essentially free of particulates. Standards for various levels of sterility (microbial limits) and particulate levels in injectable pharmaceuticals are set forth, for example, in the U.S. Pharmacopia (2012 Updates) and are hereby incorporated herein by reference.

In a further embodiment, the API is in a formulation comprising an organic solvent, an organic co-solvent and, optionally, a lipid. In a further embodiment, the organic solvent is an alcohol, the organic co-solvent is polyethylene glycol (PEG) and the lipid is tocopheryl polyethylene glycol succinate (TPGS), for example, tocopheryl succinic acid covalently linked to polyethylene glycol. In a further embodiment, the tocopherol contained in the TPGS is d-alpha tocopheryl. In a further embodiment, the TPGS is d-alpha tocopheryl PEG1000 succinate. In a further embodiment, the lipid is tocopherol covalently linked to polyethylene glycol via esterification with, citraconate, or a maleate diester linker.

In a further embodiment, the alcohol is ethanol (EtOH) and the PEG is PEG400. In a further embodiment, the formulation is in an aqueous liquid unit dosage form.

In a further embodiment, the formulation comprising an organic solvent, an organic co-solvent and, optionally, a lipid is in the following concentrations. As used herein, it is of course understood that, reference to organic solvent also includes an alcohol or EtOH, reference to an organic co-solvent also includes PEG or PEG400 and reference to a lipid also refers to TPGS. This embodiment includes an organic solvent in an amount of 1 to 20% or 3 to 15%, an organic co-solvent in an amount of 20-60% or 40-55% (vol./vol.) and a lipid in an amount of 0.1% to 30% or 1 to 20% (vol./vol.) and any integer percent contained within the foregoing ranges. In a further embodiment, the organic solvent is in an amount of about 5% or 5% (vol./vol.), the organic co-solvent is in an amount of about 40% or 40% (vol./vol.) and the lipid is in an amount of about 6% or 6% (vol./vol.).

It is understood that the above ranges of organic solvent or organic co-solvent may be higher than the above ranges, but the decision on the ranges is guided by achieving a formulation that is pharmaceutically acceptable (e.g., at pharmaceutically acceptable toxicity levels). For instance, increased amounts of the organic solvent and co-solvent, while increasing solubility, generally will also increase toxicity. It is further understood that the ranges of lipid may be higher than the above ranges, but the range is guided by desired bioavailability and solubility and an increased amount of lipid may result in decreased oral bioavailability and a decreased aqueous solubility of the API. In a further embodiment, all or a portion of the remaining volume of the unit dosage form is comprised of a pharmaceutically acceptable aqueous liquid, for example, a buffered saline solution (e.g., phosphate buffered saline (PBS)), optionally containing one or more of the excipients described herein, for example, preservatives, sweetners or flavoring.

In a further embodiment, the API is API is dissolved in the organic solvent first, prior to admixture with the organic co-solvent or the lipid.

In further embodiments, methods and unit dosage forms are described herein that provide for prolonged and enhanced levels of API administration. As can be seen from the data contained herein, the API has a rapid clearance rate from plasma and, additionally, increased tumor abatement is shown with continuous and prolonged API administration.

Accordingly, in a further embodiment, are pharmaceutical compositions and treatment and prophylactic methods, which provide a desired plasma level of API in the subject for a desired number of hours after administration. In an embodiment, the desired level of API is sustained in the subject plasma for, or for at least, 1, 1.5 (36 hours), 2, 3, 4, 5, or 6 days, 1, 2, 3 or 4 weeks, 1, 2, 3, 4, 5, 6, 7, 8, 9, 10, 11 or 12 months, 1, 2, 3, 4, 5, 6, 7, 8, 9 or 10 years or all or substantially all of the remaining life of the subject. In a further embodiment, the desired level of API is sustained in the subject plasma for, or for at least, from 1-6 days, from 1-4 weeks, from 1-10 years or for all intermediate integer ranges of time within the foregoing ranges. In this embodiment, the desired amount of API in subject plasma is an amount sufficient to reduce, for example, in the case of Ewings' Sarcoma, tumor cell growth, to reduce tumor size or to kill tumor cells. In a further embodiment, the desired amount of API in subject plasma is sustained at a level of at least 0.1, 0.2, 0.3, 0.4, 0.5. 0.6, 07, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 11, 12, 13, 14, 15, 16, 17, 18, 19 or 20 μM in subject plasma for the foregoing time durations.

The desired sustained amount of subject plasma API can be achieved, for example, by multiple daily dosings sustained for multiple days. For example, in addition to one administration per day, the pharmaceutical composition may be administered, for example, once a day, twice a day, three times a day, four times a day, five times a day, or six times a day, or at least once a day, twice a day, three times a day, four times a day, five times a day, or six times a day, with such daily administrations given for, for example, for, or for at least, 1, 1.5 (36 hours), 2, 3, 4, 5, or 6 days, 1, 2, 3 or 4 weeks, 1, 2, 3, 4, 5, 6, 7, 8, 9, 10, 11 or 12 months, 1, 2, 3, 4, 5, 6, 7, 8, 9 or 10 years or all or substantially all of the remaining life of the subject or for, or for at least, from 1-6 days, from 1-4 weeks, from 1-10 years or for all intermediate integer ranges of time within the foregoing ranges.

Moreover, in the case of an injectable administration, for example, an intravenous administration, the composition can be administered continuously for, or for at least, 1, 1.5 (36 hours), 2, 3, 4, 5, or 6 days, 1, 2, 3 or 4 weeks, 1, 2, 3, 4, 5, 6, 7, 8, 9, 10, 11 or 12 months, 1, 2, 3, 4, 5, 6, 7, 8, 9 or 10 years or all or substantially all of the remaining life of the subject or for, or for at least, from 1-6 days, from 1-4 weeks, from 1-10 years or for all intermediate integer ranges of time within the foregoing ranges.

Moreover, the compositions described herein may be administered continuously or essentially continuously (e.g., continuously except for gaps of one to seven days) for a period of months or years or for the lifetime (providing for gaps of one to 12 months) of the subject in order to act therapeutically or prophylactically.

In a further embodiment, the API is administered to a subject in a daily amount of, or in a unit dosage form of, for example in a oral dosage form, 1, 2, 3, 4, 5, 6, 7, 8, 9, 10, 11, 12, 13, 14, 15, 16, 17, 18, 19, 20, 30, 40, 50, 60, 70, 80, 90,100, 110, 120, 130, 140, 150, 160, 170, 180, 190, 200, 210, 220, 230, 240, 250, 260, 270, 280, 290, or 300 mg/kg of API per weight of the subject or at least each of the foregoing amounts or from 100 to 300 mg/kg and further including all integers between the 100-300 mg/kg range. Furthermore, included in further embodiments the API is administered in a daily amount or in a unit dosage form, for example, in an oral dosage form, for each of the foregoing API amounts for a subject having a weight of 65-75 kg or any integer between this range of 65-75kg.

In a further embodiment, the API is administered to a subject in a daily amount of, or in a unit dosage form of, for example in an injectable dosage form, for example an intravenous dosage form, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9,10, 11, 12, 13, 14, 15, 16, 17, 18, 19, 20, 21, 22, 23, 24, 25, 26, 27, 28, 29, or 30 mg/kg of API per weight of the subject or at least each of the foregoing amounts or from 10-30 mg/kg and further including all integers between the 10-30 mg/kg range. Furthermore, included in further embodiments the API is administered in a daily amount or in a unit dosage form, for example, in an injectable dosage form, for example an intravenous dosage form, for each of the foregoing API amounts for a subject having a weight of 65-75 kg or any integer between this range of 65-75kg.

In a further embodiment, the pharmaceutical composition, in an aqueous liquid unit dosage form, comprises the API in an amount of 10, 15, 20, 25, 30, 35, 40, 45, 50, 55, 65, 70, 75, 80, 85, 90, 95, 100, 110, 120, 130, 140, 150, 160, 170, 180, 190, 200 mg/mL, or at least each of the foregoing and also including from 10 mg to 30, 60, 80, 100, 125, 150, 175 or 200 mg/mL and all integers between the foregoing ranges.

### EXAMPLES

### Example 1

A Cremophor EL-Ethanol (CEL) formulation of the compound of Formula I and a Polyethylene Glycol400 formulation of the compound (PEG) of Formula I were prepared and found to have a solubility in water of 5 mg/mL.

The below graph and Table 1 show results of a pharmacokinetics data of these two formulations in CF-1 mice comparing the two formulations administered intraperitoneally (IP) with a single dose of 75 mg/kg. In the below graph, the IC₅₀ line for JARAM-2012 (abbreviated as JARAM in the graph) represents the IC₅₀ level for the compound of Formula I in cytotoxicity assays of Ewing's sarcoma family of tumors (ESFT) cell lines.

TABLE 1

| | CEL | PEG |
|---|---|---|
| Time | (μM) | (μM) |
| 0.00 | 0.0 | 0.0 |
| 0.08 | 31.1 | 34.8 |
| 0.25 | 43.1 | 33.6 |
| 0.5 | 20.0 | 20.7 |
| 1 | 2.2 | 13.1 |
| 2 | 0.3 | 5.7 |
| 4 | 0.2 | 2.3 |
| 6 | 0.1 | 2.2 |
| 8 | 0.0 | 0.7 |
| 12 | 0.0 | 0.1 |
| 24 | 0.0 | 0.0 |

### Example 2

Pharmacokinetics and bioavailability data was obtained from animal studies for a 50% PEG 400 plus 10% EtOH plus PBS formulation (Groups 1, 2 & 3) and a 40% PEG 400 plus 5% EtOH plus 18% TPGS plus PBS formulation (Groups 4, 5 & 6) and are shown below in Table 2.

The following abbreviations are used in Table 1:

IV=intravenous administration

PO=oral administration

AUC=area under the curve

CL=clearance

lz (lambda z)=first order rate constant associated with the terminal (log-linear) portion of; the curve

HL lz (t½): Terminal half-life

Vd=volume of distribution based on the terminal phase

Vss=steady state volume of distribution.

TABLE 2

| | | 2038-7 | | | | | |
|---|---|---|---|---|---|---|---|
| | | Grp 1 | Grp 2 | Grp 3 | Grp 4 | Grp 5 | Grp 6 |
| IV Parameters | PO Parameters | (IV) | (PO) | (PO) | (IV) | (PO) | (PO) |
| Dose, mg/Kg | Dose, mg/Kg | 25 | 25 | 150 | 40 | 40 | 400 |
| AUC, hr · ng/mL | AUC, hr · ng/mL | 6,157.0 | 4,516.9 | 22,572.4 | 46,858.8 | 4,504.8 | 62,531.7 |
| CL, mL/hr/Kg | CL/F, mL/hr/Kg | 4,060.4 | 5,534.8 | 6,645.3 | 853.6 | 8,879.4 | 6,396.8 |
| lz (k), hr−1 | lz (k), hr−1 | 0.1 | 0.2 | 0.2 | 0.1 | 0.3 | 0.4 |
| HL lz (t½), hr | HL lz (t½), hr | 5.1 | 2.8 | 4.3 | 4.7 | 2.1 | 1.9 |
| Vd (=Vz), mL/Kg | Vz/F, mL/Kg | 29,620.5 | 22,163.5 | 40,750.0 | 5,837.0 | 26,332.8 | 17,959.4 |
| Vss, mL/Kg | Vss, mL/Kg | 9,841.4 | 5,582.9 | 33,268.7 | 1,043.4 | 28,723.5 | 32,859.6 |

### Example 3

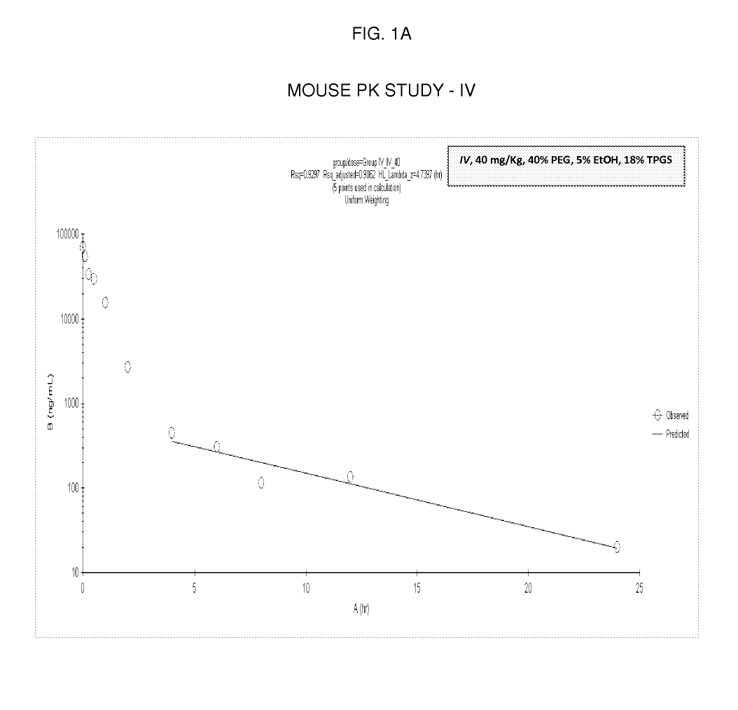

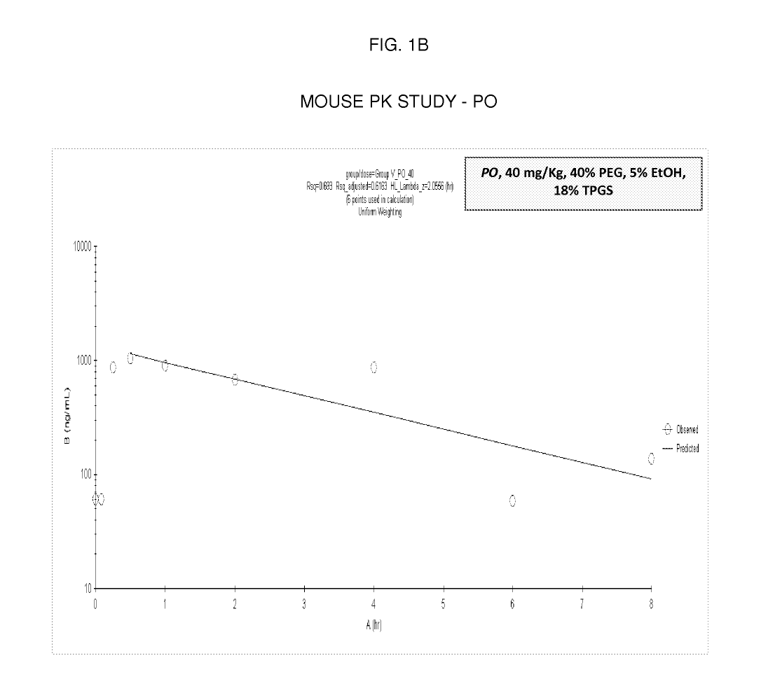

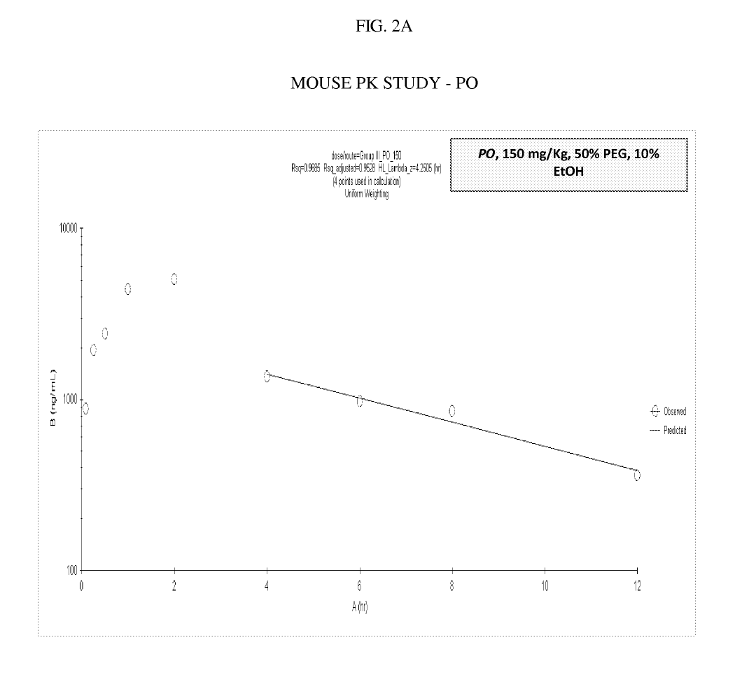

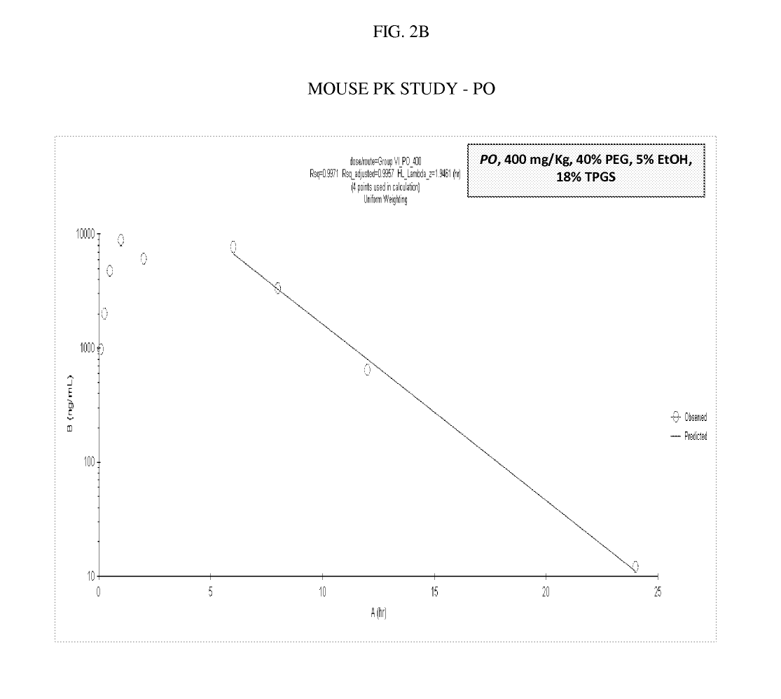

The API was administered to CF1 mice. Blood was drawn from the mice at predetermined time intervals. Plasma was isolated from the blood and analyzed using LCMS to determine amounts of API (ng/mL). The data shown in FIG. 1A was obtained for IV administration of 40 mg/kg of API in a formulation containing 40% PEG400, 5% EtOH and 18% TPGS (d-alpha tocopheryl PEG1000 succinate). The data shown in FIG. 1B was obtained for PO administration of 40 mg/kg of API in a formulation containing 40% PEG400, 5% EtOH and 18% TPGS (d-alpha tocopheryl PEG1000 succinate). The data shown in FIG. 2A was obtained for PO administration of 150 mg/kg of API in a formulation containing 50% PEG400 and 10% EtOH. The data shown in FIG. 2B was obtained for PO administration of 400 mg/kg of API in a formulation containing 40% PEG400, 5% EtOH and 18% TPGS (d-alpha tocopheryl PEG1000 succinate). Time 0 in FIGS. 1A, 1B, 2A and 2B represent the time of administration of the API.

### Example 4

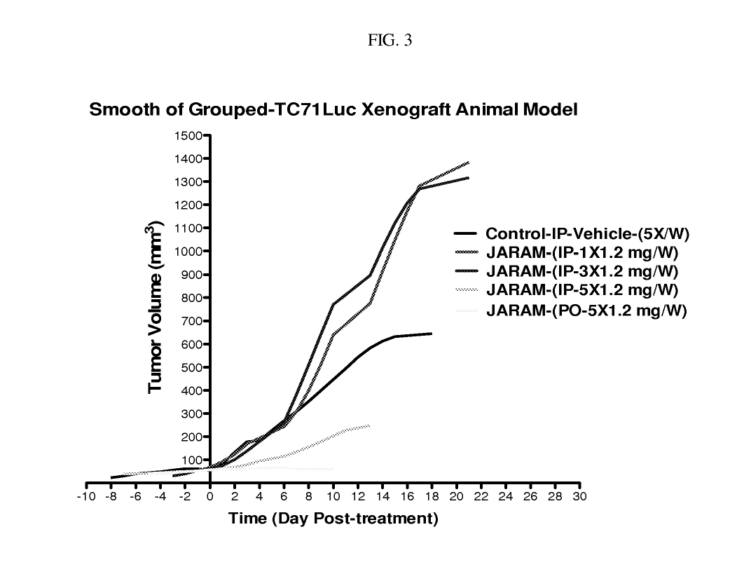

Using a TC71Luc Ewing's sarcoma xenograft murine model, mice were subcutaneously injected with 1 million TC71Luc cells. At a predetermined tumor volume (time=0), mice were given the first administration of API in an EtOH (5%), PEG400 (40%), PBS formulation. FIG. 3 depicts data obtained from control and for administration once per week (1×), three times per week (3×) and five times per week (5×) in each case at a dosage of 1.2 mg per mouse.

### Example 5

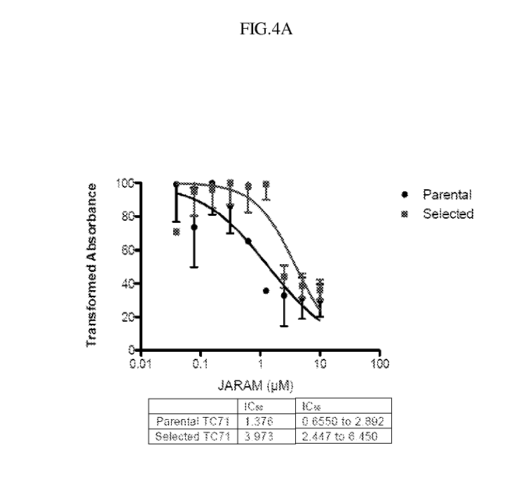

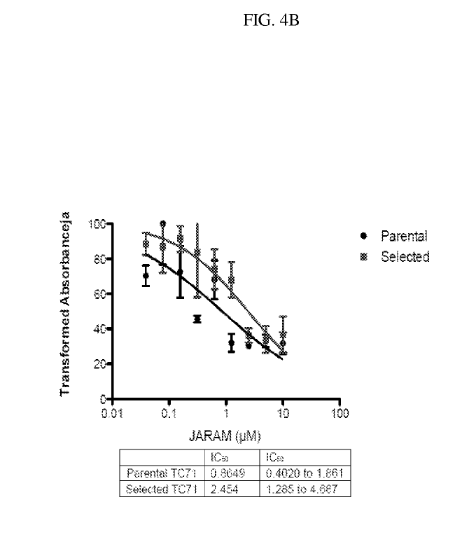

In vitro cytotoxicity assays were used with TC71Luc Ewing's sarcoma cell lines in the presence of the compound of Formula I in an EtOH (5%), PEG400 (40%) formulation. IC₅₀ values were obtained via absorbance measurements indicating the amount of viable cells. IC₅₀ values are shown in the tables and graphs in FIGS. 4A and 4B.

1. A pharmaceutical composition, comprising the compound of Formula I: 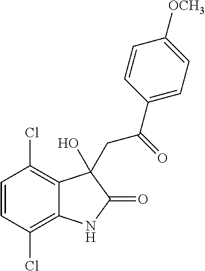 [SMILES: COc1ccc(C(=O)CC2(O)C(=O)Nc3c(Cl)ccc(Cl)c32)cc1]
or a pharmaceutically acceptable salt thereof in an amount effective to treat Ewing's Sarcoma in a subject in need thereof, and further comprising an organic solvent, an organic co-solvent and a lipid in a pharmaceutically acceptable aqueous liquid unit dosage form.

2. The composition of claim 1, wherein the organic solvent is ethanol, the organic co-solvent is polyethylene glycol (PEG) and the lipid is tocopheryl polyethylene glycol succinate (TPGS).

3. The composition of claim 1, wherein the organic co-solvent is PEG 400 and the lipid is d-alpha tocopheryl PEG1000 succinate.

4. The composition of claim 2, wherein the PEG is in an amount of 40-50% (vol./vol.).

5. The composition of claim 2, wherein the ethanol is in an amount of 5-15% (vol./vol.).

6. The composition of claim 2, wherein the TPGS is in an amount of 0.1% to 20% (vol./vol.).

7. The composition of claim 3, wherein the ethanol is in an amount of about 5% (vol./vol.), the PEG 400 is in an amount of about 40% (vol./vol.) and the d-alpha tocopheryl PEG1000 succinate is in an amount of about 6% (vol./vol.).

8. A method for treatment or prophylaxis of Ewing's sarcoma in a subject in need thereof, comprising administering to the subject at least once a day for at least 1.5 days, the compound of Formula I or a pharmaceutically acceptable salt thereof.

9. The method of claim 8, wherein the compound of Formula I is continuously administered intravenously for at least 36 hours.

10. The method of claim 8, wherein the compound of Formula I is administered at least once a day for at least 2 days.

11. The method of claim 8, wherein the compound of Formula I is administered at least once a day for at least 3 days.

12. The method of claim 8, wherein the compound of Formula I is administered at least once a day for at least 5 days.

13. The method of claim 8, wherein the compound of Formula I is administered at least once a day for at least 7 days.

14. The method of claim 8, wherein the compound of Formula I is administered orally.

15. A method for treatment or prophylaxis of Ewing's sarcoma in a subject in need thereof, comprising administering to the subject the compound of Formula I or a pharmaceutically acceptable salt thereof such that plasma levels of at least 1 micromolar (μM) of the compound of Formula I are sustained in the subject's plasma for at least 36 hours.

16. The method of claim 15, wherein the compound of Formula I is administered orally.

17. The method of claim 15, wherein the compound of Formula I is administered intravenously.
